(19) United States
(12) Patent Application Publication
(10) Pub. No.: US 2016/0129068 A1
(43) Pub. Date: May 12, 2016

(54) COMPOSITION OF PLANT EXTRACT AND ITS PHARMACEUTICAL COMPOSITION AND APPLICATION THEREOF

(71) Applicant: Caliway Biomedical Co., Ltd., Taipei (TW)

(72) Inventor: Yu-Fang Ling, Taipei (TW)

(21) Appl. No.: 14/536,741

(22) Filed: Nov. 10, 2014

(51) Int. Cl. A61K 36/9066; A61K 31/353; A61K 31/047

(52) U.S. Cl. CPC A61K 36/9066; A61K 31/047; A61K 31/353

(57) ABSTRACT

The present invention is related to a composition for relieving joint inflammation. The present invention is also related to a method for treatment of joint inflammation, comprising a step of administering to a subject in need thereof a therapeutically effective amount of a pharmaceutical composition. By means of inhibiting the COX-2 and NF-κB expression through the composition or the pharmaceutical composition with specific ratio, the joint inflammation and swelling could be reduced and the osteoclasts proliferation could be decreased to mitigate bone erosion, osteoporosis and degenerative arthritis.

### BACKGROUND OF THE INVENTION

1. Field of the Invention

The present invention relates to a composition obtained from plant extract, and more particularly the use of the composition is for relieving arthritis symptoms.

2. Description of the Prior Arts

Degenerative arthritis may occur in any joint of the body, wherein the most frequently region is knee joint and hip joint, and degenerative arthritis mostly occur among the elderly, athletes or people with obesity. Even age and obesity are major risk factors of degenerative arthritis, the pathogenesis of the degenerative arthritis is still unknown. Modern people may suffer from some degenerative arthritis in compliance with the increasing age due to extended life span, so that patients suffer swelling and pain, even functional limitations or disability under long time threat of degenerative arthritis.

According to statistics, the global prevalence of degenerative arthritis is approximately 11.3%, and the number of people worldwide who suffer from degenerative arthritis was 8.5 million in 2009 and will continuously increase to 120 million in 2017. Moreover, there are nearly 70 percent elders suffer from degenerative arthritis, and the ages of pathogenesis have been progressively younger in trend. The prevalence of degenerative arthritis in men is 9.6% and in women is 18% in the population aged over 60. The proportion of women suffering degenerative arthritis is two times of men because women have wider pelvis and have lesser leg muscle than men. World Health Organization (WHO) further predicts more than 25% of the population will suffer degenerative arthritis in 2025, wherein among population aged over 65 the percentage will be 74%. The number of patients suffering from degenerative arthritis is about 1.58 million in the United States, and there are up to 79% elders suffer from pain caused from degenerative arthritis and disability. There are 40% people over age 40 also suffering degenerative arthritis in, and as estimated, more than 70 million people will suffer degenerative arthritis in 2030 in the United States. Degenerative arthritis has become the second major factor of unemployment in the United States, and the medical expenses caused by degenerative arthritis accounted for 2.5% of the gross production of the United States, and then lead to 65 billion dollars in the annual impact on the economic and social costs. Thus, degenerative arthritis cannot be ignored as an important chronic disease.

Degenerative arthritis may be caused by many risks, besides age, which is major risk, other risks also include occupational, dietary habits, obesity, and bone density. Diagnosis for this disease is usually through clinical observation and X-ray to indentify this disease. Based on the damage of the cartilage, degenerative arthritis can be divided into four stages: in the first stage, the surface of the cartilage is worn, and the joint space becomes narrow via X-ray observation, and then joint pain occurs; the second phase, the cartilage is seriously worn, but does not crack; the third phase, the cartilage is cracked, and the cartilage becomes uneven; the fourth stage, the appearance of the joint becomes deformed, so that the cartilage has been unusable.

Regarding the pathogenesis of degenerative arthritis, there could be observed that synovial cavity is inflamed and associated with the loss of cartilage; even the patient would suffer swelling and deformation limbs in more severe arthritis with infiltration in cartilage and synovial tissue and full of a large number of macrophages. Therefore, various cytokines can be detected such as TNF-α, NF-κB in lesions. In particular, COX-2 is an inducible enzyme induced by inflammation. Most organizations do not have COX-2 expression in normal physiological conditions; however, a lot of COX-2 would be generated when cells are stimulated by inflammation factor such as IL-1, TNF-α, NF-κB, so as to produce prostaglandins involved inflammation, and to cause pain in joint tissue. In addition, arthritis is in compliance with abnormal proliferation of synovial cells and production of a lot of RANKL (receptor for activation of nuclear factor kappa B ligand). As RANKL is combined with RANK on the surface of macrophages, it will stimulate macrophage to differentiate into osteoclasts, and then the minerals will be released from bone calcium, eventually resulting in the loss of hard bone.

Currently, there is no way to thoroughly cure degenerative arthritis but by means of preservation and drugs to effectively control disease and relieve pain. Treatment would be administrated according to the severity of the disease, wherein the treatment comprises analgesic drug with anti-inflammatory effect or hyaluronic acid injections. If the patient cannot endure any more pain caused from knee joint even by the above therapy, the artificial joint replacement surgery is necessary to relieve symptoms. In addition, it is effective in reducing risk factors through diet and lifestyle changes, keep exercising, and weight control.

The analgesic drugs used for relieving arthritis comprise acetaminophen, nonsteroidal anti-inflammatory drugs (NSAIDs), and cyclooxygenase-2 (COX-2) inhibitors. Acetaminophen can be used to first-line drug to relieve pain and to inhibit the secretion of prostaglandins. However, prostaglandin is related to blood pressure regulation, blood clotting, gastric acid secretion and renal function regulation, so as to interfere with prostaglandins to affects the normal function. On the other hand, overdose Acetaminophen will increase the risk of gastrointestinal bleeding, and be prone to liver toxicity. NSAIDs are used to inhibit COX-1 and COX-2 and have anti-inflammatory and analgesic effects. When PGI2 reduction due to COX-2 inhibited results in anti-inflammatory analgesic effect, COX-1 inhibition would damage the integrity of the gastrointestinal mucosa and affect renal blood flow, and then lead to side effects such as gastric ulcers or kidney function decline. The COX-2 inhibitor is used to inhibit prostaglandin through COX-2 inhibition, so as to achieve inflammation and pain reduction, and to reduce nearly 50% gastric perforation, bleeding, and ulcers. However, recently studies show that COX-2 inhibitor causes cardiovascular toxicity and risk, so the COX-2 inhibitor is recommended for use for patients with high gastrointestinal tract risk and low cardiovascular risk.

In summary, the conventional drugs used to treat degenerative arthritis only suppress pain, but those drugs cannot treat the pathogenesis or relieve the pain from the origin; those drugs not only cause liver toxicity, gastrointestinal bleeding, ulcers, kidney failure and other side effects, but also are a big burden to the elders, the major group suffering degenerative arthritis. Moreover, even COX-2 inhibitors with specificity analgesic have better analgesic effect, they would cause cardiovascular toxicity, have no significantly improvement among the elders who have severe joint wear, and the cardiovascular toxicity to the elders is a big risk. Glucosamine has no apparent efficiency for analgesic and anti-inflammatory, especially for the patient of higher age. Regarding hyaluronic acid joint injections, patients have to receive injections once a week and continuously for more than five weeks, resulting in increasing discomfort and risk of infection. The function of hyaluronic acid joint injections is to increase joint lubrication to relieve pain, but that cannot directly reduce inflammation factor. Conventional drugs for treating degenerative arthritis drugs are only temporary treatment.

### SUMMARY OF THE INVENTION

To overcome the shortcomings of the lack of a drug capable of anti-inflammation, reducing pain and side effects, and being safer for treating degenerative arthritis, the objective of the present invention is to provide a composition for use in relieving joint inflammation comprising: turmeric extract and at least one plant extract selected from the group consisting of resveratrol, quercetin and the combination thereof. In another preferred embodiment, the composition comprises turmeric extract and resveratrol, wherein a weight ratio of turmeric extract to resveratrol is between 1:4 and 49:1.

In another preferred embodiment, the composition comprises turmeric extract and quercetin, wherein a weight ratio of turmeric extract to quercetin is between 2:3 and 48:1.

In another preferred embodiment, the composition comprises turmeric extract, resveratrol and quercetin, wherein a weight ratio of turmeric extract to resveratrol to quercetin is between 18:1:1 and 5:1:4.

According to the present invention, the term “turmeric extract” as used herein mainly comprises curcumins. Preferably, the amount of the curcumins ranges from 90% to 100% of the total amount of the turmeric extract.

According to the present invention, the term “joint”, as used herein, refers to a junction between two bones or more than two bones with activity. Preferably, the joint includes, but is not limited to, knee joint and hip joint.

According to the present invention, the term “joint inflammation”, as used herein, refers to cartilage degeneration or connective tissue inflammation, and the symptoms are joint pain. The disease related to “joint inflammation” includes, but is not limited to, osteoarthritis, rheumatoid arthritis, traumatic arthritis and septic arthritis.

The present invention further provides a pharmaceutical composition, wherein the pharmaceutical composition comprises a therapeutically effective amount of the above composition and a pharmaceutically acceptable carrier.

According to the present invention, the term “therapeutically effective amount” as used herein, refers to a dosage to inhibit or alleviate joint inflammation. The therapeutically effective amount for inhibiting or alleviating joint inflammation is determined by administering the pharmaceutical composition in an effective amount, and measuring the COX-2 and NF-κB expression in a specific period.

According to the present invention, the term “pharmaceutically acceptable carriers” as used herein includes any physiologically compatible and all solvents, dispersion medium, antibacterial and antifungal agents, isotonic and absorption delaying agents and analogues thereof. For example, the pharmaceutically acceptable carriers include one or more water and combination of water, salt water, phosphate buffered saline (PBS), dextrose, glycerol, ethanol and its analogues. Preferable combination includes isotonic agents, for example, sugar or polyol such as mannitol, sorbitol, or sodium chloride. The pharmaceutically acceptable carriers further include microscale auxiliary substances such as wetting or emulsifying agents, preservatives or buffers.

In accordance with the present invention, the pharmaceutical composition for relieving joint inflammation is prepared for multiple forms, includes, but is not limited to, liquid, semi-solid and solid dosage, and such as liquid solution (including injectable and infusible solution), dispersions or suspensions, tablets, pills, powders, liposomes and suppositories. Preferred form depends on the mode of administration and therapeutic application of expectations. Preferably, the pharmaceutical composition of the present invention is administered orally or in the form of infusion solutions, and the preferred mode of administration is enteral modes, such as orally. In an embodiment of the present invention, the pharmaceutical composition at the effective amount is orally administrated. Preferably, the pharmaceutical composition of the present invention is administered in the form of injection. More preferably, the form of injectable administration is selected from the group consisting of solution and suspension.

The present invention further provides a method for relieving joint inflammation comprising a step of administering to a subject in need thereof a therapeutically effective amount of a pharmaceutical composition in the effective amount and a pharmaceutically acceptable carrier.

Preferably, the therapeutically effective amount of the pharmaceutical composition is between 0.1 mg/kg/day and 67 mg/kg/day.

The present invention further provides a method for preparing said pharmaceutical composition comprising resveratrol and turmeric extract in a specific amount including a step of mixing a composition comprising resveratrol and turmeric extract and pharmaceutically acceptable salts, pharmaceutically acceptable stabilizers or pharmaceutically acceptable excipients.

According to the present invention, the term “pharmaceutically acceptable salts” as used herein includes any physiologically compatible and all solvents.

According to the present invention, the term “pharmaceutically acceptable stabilizers” as used herein is a stabilized pharmaceutical composition as defined, such as stabilizers, includes, but is not limited to, inorganic alkaline and alkaline earth metal salts, oxides and hydroxides.

According to the present invention, the term “pharmaceutically acceptable excipients” as used herein includes, but is not limited to, disintegrants, binders, fillers, lubricants and glidants. The amount of excipient employed will depend upon how much active agent is to be used. One excipient can perform more than one function. Examples of disintegrants include, but are not limited to, agar-agar, alginic acid, calcium carbonate, carboxymethylcellulose, cellulose, clays, colloidal silica, croscarmellose sodium, cross-linked povidone, gum, silicon magnesium aluminometasilicate, methyl cellulose, polacrilin potassium, sodium alginate, low substituted hydroxypropyl cellulose, and crosslinked polyvinylpyrrolidone hydroxypropylcellulose, sodium starch glycolate, and starch. Examples of binders include, but are not limited to, microcrystalline cellulose, hydroxymethyl cellulose, hydroxypropyl cellulose and polyvinyl pyrrolidone. Examples of fillers include, but are not limited to, calcium carbonate, calcium phosphate, dibasic calcium phosphate, tribasic calcium sulfate, calcium carboxymethylcellulose, cellulose, dextrin, salt, dextrin, dextrose, fructose, lactitol, lactose, carbonate, magnesium oxide, maltitol, maltodextrin, maltose, sorbitol, starch, sucrose, sugar, and xylitol. Examples of lubricants include, but are not limited to, agar, calcium stearate, ethyl oleate, ethyl laureate, glycerin, glyceryl palmitostearate, hydrogenated vegetable oil, magnesium oxide, magnesium stearate, mannitol, poloxamer, ethylene glycol, sodium benzoate, sodium lauryl sulfate, sodium stearyl acid, sorbitol, stearic acid, talc and zinc stearate. Examples of glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

By means of inhibiting the expression of the inflammation factors NF-κB and COX-2 through the composition or the pharmaceutical composition in accordance with the present invention, the joint inflammation and swelling can be reduced and the osteoclasts proliferation can be decreased for the degenerative arthritis patients to mitigate bone erosion and osteoporosis. The composition or pharmaceutical composition in accordance with the present invention not only can inhibit inflammation at bone joint to avoid significant side effects caused by conventional drugs, but also can inhibit macrophage to differentiate into osteoclasts at bone joint to reduce the risk of bone erosion and osteoporosis. Besides, the composition or the pharmaceutical composition in accordance with the present invention has no significant side effects for the elders, the major group suffering degenerative arthritis. Therefore, the composition or the pharmaceutical composition in accordance with the present invention differs from used analgesic, anti-inflammatory drugs or hyaluronic acid injections.

### BRIEF DESCRIPTION OF THE DRAWINGS

FIG. 1 illustrates the quantified bar chart in each group for inhibiting COX-2 mRNA expression;

FIG. 2 illustrates the quantified bar chart in each group for inhibiting NF-κB mRNA expression; and,

FIG. 3 illustrates the quantified bar chart and the electrophoresis result via western blot assay in each group for inhibiting COX-2 protein expression.

### DETAILED DESCRIPTION OF THE PREFERRED EMBODIMENTS

Other objectives, advantages and novel features of the invention will become more apparent from the following detailed description when taken in conjunction with the accompanying drawings.

### Example 1

### Inflammation Factor COX-2 and NF-κB Gene Expression Assay

About 3×10⁵ RAW264.7 cells obtained from Food Industry Research and Development Institute (FIRDI, Taiwan) were seeded in 6 cm dishes under 37° C., 5% CO₂ for 24 hours. After 24 hours, medium was removed and washed by PBS, and then 10 ppm control group (DMSO), 10 ppm LPS group, 10 ppm quercetin group, 10 ppm resveratrol group, 10 ppm turmeric extract group and the composition as OT-001-R1, OT-001-R2, OT-001-Q, OT-001-C1, OT-001-C2, OT-001-C3, OT-001-C4, and OT-001-05 in accordance with the present invention all were 30 ppm and respectively added to the dishes, wherein the composition OT-001-R1 in accordance with the present invention has a weight ratio of resveratrol to turmeric extract to quercetin being 4:1:0; the composition OT-001-R2 in accordance with the present invention has a weight ratio of resveratrol to turmeric extract to quercetin being 3:1:1; the composition OT-001-Q in accordance with the present invention has a weight ratio of resveratrol to turmeric extract to quercetin being 3:0:2; the composition OT-001-C1 in accordance with the present invention has a weight ratio of resveratrol to turmeric extract to quercetin being 1:4:0; the composition OT-001-C2 in accordance with the present invention has a weight ratio of resveratrol to turmeric extract to quercetin being 0:2:3; the composition OT-001-C3 in accordance with the present invention comprises a weight ratio of resveratrol to turmeric extract to quercetin being 0:3:2; the composition OT-001-C4 in accordance with the present invention has a weight ratio of resveratrol to turmeric extract to quercetin being 0:4:1; the composition OT-001-05 in accordance with the present invention has a weight ratio of resveratrol to turmeric extract to quercetin being 2:3:0; all groups were repeatedly tested for 3 time.

After incubation for 3 hours, except for control group (DMSO), the other groups were then added with 100 ng/ml LPS to induce inflammation; after incubation for 3 hours, the medium containing LPS was drained and rinsed with PBS. Then, 1 ml TRIzol RNA was respectively added for RNA extraction, and then the COX-2 and NF-κB gene expression were respectively analyzed.

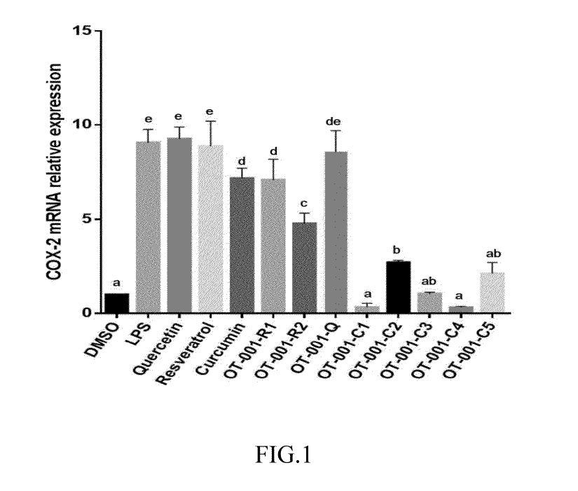

As shown in FIG. 1, compared to the control group and plant extract groups, the compositions of OT-001-R1, OT-001-R2, OT-001-C1, OT-001-C2, OT-001-C3, OT-001-C4 or OT-001-05 in accordance with the present invention could effectively inhibit COX-2 expression and had significant differences, wherein the compositions OT-001-C1 and OT-001-C4 both had the best inhibition effect.

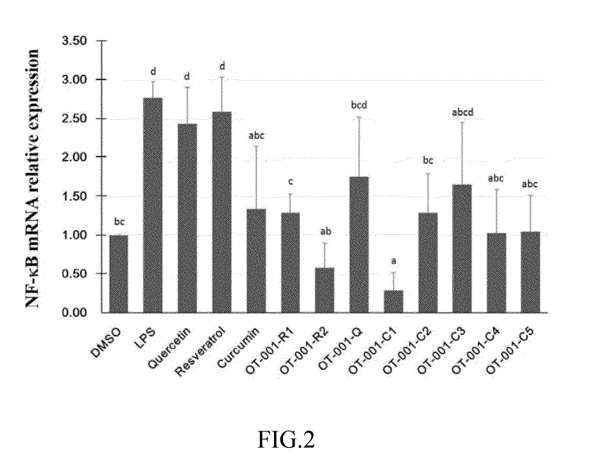

As shown in FIG. 2, turmeric extract group was capable of inhibiting NF-κB expression. Each of the compositions of OT-001-R1, OT-001-R2, OT-001-C1, OT-001-C2, OT-001-C4, and OT-001-05 all were able to inhibit NF-κB expression significantly, wherein the compositions of OT-001-C1 had the best inhibitory effect.

### Example 2

### Inflammation Factor COX-2 Protein Expression Assay

About 3×10⁵ RAW264.7 cells obtained from FIRDI were seeded in 6 cm dishes under 37° C., 5% CO₂ for 24 hours. After 24 hours, medium was removed and washed by PBS, and then 10 ppm control group (DMSO), 10 ppm LPS group, 10 ppm quercetin group, 10 ppm resveratrol group, 10 ppm turmeric extract group and the composition as above said OT-001-R1, OT-001-R2, OT-001-Q, OT-001-C1, OT-001-C2, OT-001-C3, OT-001-C4, OT-001-05 and OT-001-C6 in accordance with the present invention all were 30 ppm and respectively added to the dishes, wherein the composition OT-001-C6 has a weight ratio of resveratrol to turmeric extract to quercetin being 1:48:0; all groups were repeatedly tested for 3 time.

After incubation for 3 hours, except for control group (DMSO), the other groups were then added with 100 ng/ml LPS to induce inflammation; after incubation for 3 hours, the medium containing LPS was drained and rinsed with PBS. Then, each group proteins were respectively extracted, and western blot was used for observing COX-2 protein expression. Equal amounts of protein (25 μg) were separated by SDS-polyacrylamide gel electrophoresis and transferred to a PVDF membrane; antibody for western blot was COX-2 antibody (C-20): sc-1745 obtained from Santa Cruz.

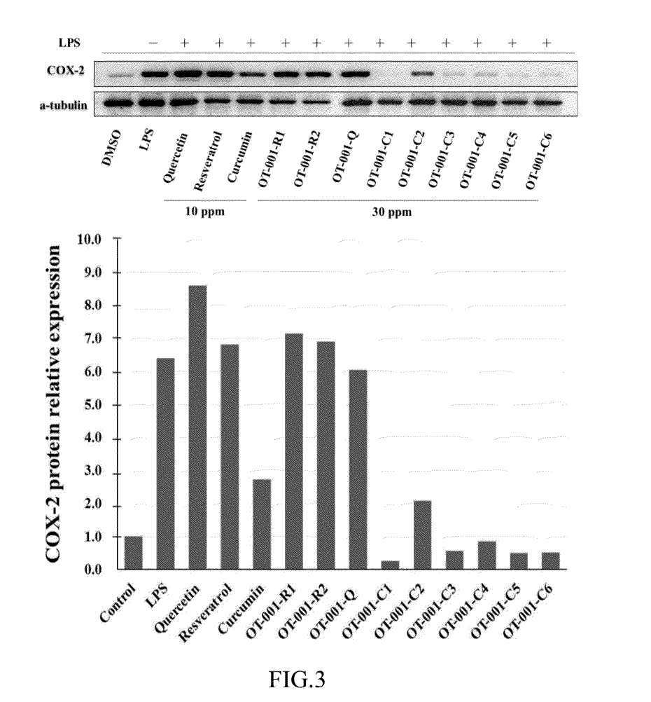

As shown in FIG. 3, the result of COX-2 inhibition was similar to RNA assay: turmeric extract group was capable of inhibiting COX-2 protein expression. Each of the compositions of OT-001-C1, OT-001-C3, OT-001-C4, OT-001-05 and OT-001-C6 all were able to inhibit COX-2 protein expression significantly.

What is claimed is:

1. A composition for use in relieving joint inflammation comprising: turmeric extract and at least one plant extract selected from the group consisting of resveratrol, quercetin and the combination thereof.

2. The composition of claim 1, wherein a weight ratio of turmeric extract to resveratrol is between 1:4 and 49:1.

3. The composition of claim 1, wherein a weight ratio of turmeric extract to quercetin is between 2:3 and 48:1.

4. The composition of claim 1, wherein a weight ratio of turmeric extract to resveratrol to quercetin is between 18:1:1 and 5:1:4.

5. The composition of claim 1, wherein the joint is knee joint or hip joint.

6. The composition of claim 1, wherein the joint inflammation is osteoarthritis, rheumatoid arthritis, traumatic arthritis or septic arthritis.

7. The composition of claim 1, wherein the composition is designed for oral or injectable administration.

8. A pharmaceutical composition, wherein the pharmaceutical composition comprises a therapeutically effective amount of the composition as claimed in claim 1 and a pharmaceutically acceptable carrier.

9. The pharmaceutical composition of claim 8, wherein the therapeutically effective amount composition for relieving joint inflammation is between 0.1 mg/kg/day and 67 mg/kg/day.

10. The pharmaceutical composition of claim 8, wherein the pharmaceutical composition is designed for oral or injectable administration.

11. The pharmaceutical composition of claim 8, wherein the form of oral administration is selected from the group consisting of solid dosage form, solution, tablet including controlled-release tablet, and capsule.

12. The pharmaceutical composition of claim 8, wherein the form of injectable administration is selected from the group consisting of solution and suspension.

13. A method for treatment of joint inflammation, comprising a step of administering to a subject in need thereof a therapeutically effective amount of the pharmaceutical composition as claimed in claim 6.

15. The method of claim 13, wherein the joint inflammation is osteoarthritis, rheumatoid arthritis, traumatic arthritis or septic arthritis.

16. The method of claim 13, wherein the therapeutically effective amount of the pharmaceutical composition for relieving joint inflammation is between 0.1 mg/kg/day and 67 mg/kg/day.

17. A method for preparing the pharmaceutical composition as claimed in 8 including a step of mixing a composition as claimed in 1 and pharmaceutically acceptable salts, pharmaceutically acceptable stabilizers or pharmaceutically acceptable excipients for oral or injectable administration.

18. The method of claim 17, wherein the form of oral administration is selected from the group consisting of solid dosage form, solution, tablet including controlled-release tablet, and capsule.

19. The method of claim 17, wherein the form of injectable administration is selected from the group consisting of solution and suspension.
